(19) United States
(12) Patent Application Publication
(10) Pub. No.: US 2020/0234792 A1
(43) Pub. Date: Jul. 23, 2020

(54) METHOD FOR OBTAINING MICROORGANISM INFORMATION USING TETRA-NUCLEOTIDE FREQUENCY

(71) Applicant: CHUNLAB, INC., Seoul (KR)

(72) Inventor: Sung-Min HA, Yongin-si (KR)

(21) Appl. No.: 16/487,598

(22) PCT Filed: Feb. 22, 2018
(86) PCT No.: PCT/KR2018/002204

§ 371 Date: Dec. 24, 2019

(30) Foreign Application Priority Data
Feb. 22, 2017 (KR) 10-2017-0023725

(51) Int. Cl. G16B 30/00; G16B 25/00; G16B 40/00

(52) U.S. Cl. CPC G16B 30/00; G16B 40/00; G16B 25/00

(57) ABSTRACT

The present invention relates to a method for obtaining reference microorganism information for genome analysis of a sample microorganism by analyzing a tetra-nucleotide frequency of the sample microorganism.

### TECHNICAL FIELD

The present invention relates to a method for obtaining microorganism information using a tetra-nucleotide frequency (TNF), and relates to a method for obtaining reference microorganism information for genome analysis of a sample microorganism, comprising obtaining information of the reference microorganism selected based on correlation of genome TNFs, by comparing genome TNFs of the unknown sample microorganism and the reference microorganism.

### BACKGROUND ART

Developments of the Next Generation Sequencing (NGS) have made genome sequencing much easier with relatively less cost and time when compared to the conventional Sanger sequencing technology. With the completion of the human genome, many other organisms were targeted for genome sequencing. Among these organisms, there has been an exponential growth in microbial genomes due to their simplicity and small size of the genome.

As a result of prevalence in genome data, the comparison of genome data has unveiled the hidden features such as symbiotic relationship, pathogenic status, favorable habitats, or the state of evolution. The burden of comparative genomics has decreased drastically with the advancement in bioinformatics algorithms and computer technology, which were previously noted as a labor-intensive and complicated task. It is only a matter of time that this comparative analysis will be one of the essential steps in genomic analysis. A suitable set of genomes must be made based on certain aspects such as sampling site, the existence of a specific gene(s), pathogenicity, and the like. However, the process of finding such features are frequently considered to be a time-consuming and challenging task. Thus, the current invention involves the software tools that can automate the generation of CG set using a combination of TNF and single nucleotide polymorphism (SNP) to find similar genome in the reference database and related metadata for the microorganism (bacteria) of interest. Although TNF and SNP are widely used in metagenome and genome analysis, they never have been used in the generation of CG set as a search tool.

Specifically, one of the critical aspects of the current invention involves the provision of epidemiologically relevant information in an urgent situation like an outbreak. The information provided may be used in the identification of pathogenicity, fast and accurate diagnostics or treatment of patients, finding a source of infection, etc. In the past, researchers had to find numerous research articles of pathogens manually to find relevant information to compare. Also, the false result may have aroused due to different analytical methods exploited for analysis of each genome.

### DISCLOSURE

### Technical Problem

In order to solve such problems, the present invention constructs database related to a genome including at least one or more kinds of microorganism genomes and constructs a system for finding a genome similar to genome of a sample microorganism rapidly and accurately using a tetra-nucleotide frequency or an SNP, and thereby provides a method for obtaining information of a reference microorganism for genome analysis of a sample microorganism or a computer readable method for obtaining information of a reference microorganism for genome analysis of a sample microorganism.

An embodiment of the present invention is to provide a method for obtaining reference microorganism information for genome analysis of a sample microorganism, comprising selecting the reference microorganism having high correlation coefficient with a sample microorganism to be analyzed, by using a whole genome nucleotide sequence and a genomic TNF of a microorganism.

An embodiment of the present invention is to provide a method of tracking down rapidly a pathogenic strain using a whole genome nucleotide sequence and a genomic TNF of a microorganism.

Another embodiment of the present invention is to provide a method for analyzing a core gene, a genomic island or a pan-genome of a sample microorganism, using the information of a reference microorganism for genome analysis of a sample microorganism.

Other embodiment of the present invention is to provide a storage medium recording a computer readable program for executing a method for obtaining information of a reference microorganism for genome analysis of a sample microorganism using the method for obtaining information of a reference microorganism for genome analysis of a sample microorganism.

### Technical Solution

An embodiment of the present invention relates to a method for obtaining a reference microorganism for analyzing a genome of the sample microorganism, wherein the method comprises:

providing a genomic tetra-nucleotide frequency (TNF) of the reference microorganism,

comparing a genome TNF of the sample microorganism and the reference microorganisms, and selecting reference microorganism based on correlation value of their TNFs, and,

obtaining information of the selected reference microorganisms.

The step of selecting the reference microorganism based on correlation coefficient value of the TNF by comparing genomic TNFs of the sample microorganism and the reference microorganism is performed based on the order of high correlation coefficient of the genomic TNF of the reference microorganisms to the genomic TNF of the sample microorganism.

Another embodiment of the present invention relates to a computer reading method for obtaining information of reference microorganisms being useful for analyzing the genome of a sample microorganism, using the method for obtaining information of a reference microorganism for analyzing the genome of a sample microorganism.

Other embodiment of the present invention relates to a storage medium recording a computer readable program for executing a method for obtaining information of a reference microorganism for analyzing the genome of a sample microorganism, using the method for obtaining information of a reference microorganism for analyzing the genome of a sample microorganism.

An embodiment of the present invention provides a method of rapidly tracing a pathogenic strain, by identifying pathogenicity of a sample microorganism using a genome nucleotide sequence and a genomic TNF of a microorganism. Specifically, the present invention relates to a method for rapidly tracking dawn the pathogenic microorganism using other pathogenic microorganism similar to the pathogenic microorganism in a sample as a reference microorganism, by analyzing TNF and/or an SNP of the sample pathogenic microorganism. The metadata information of the selected reference microorganisms, for example, at least one information selected from the group consisting of taxonomically valid name, information about phylogenetic similarity, information about the pathogenicity, environmental information on source, year of discovery, country of discovery, and related literature information for the selected reference microorganisms can be obtained, so as to track down the pathogenicity of the sample microorganism by utilizing information of the reference microorganism.

More specifically, in the present invention, the step of obtaining information on the selected reference microorganism may be performed by aligning the whole genome nucleotide sequence of the selected reference microorganism to analyze the number of SNP regions and providing information related to phylogenetic similarity of the selected reference microorganism to a sample microorganism, based on the number of SNP regions. The information may be utilized for tracking the sources of strains in addition to identifying strains.

Herein, the step of tracking down a pathogenic strain includes identifying the temporal and geographic migration and the evolutionary pathways of microorganisms, by performing phylogenetic similarity analysis with an SNP between the reference microorganisms selected by using the TNF and the sample microorganism, and other additional information. When an event such as bacterial outbreak occurs, the rapid diagnosis is required for increasing the survival rate of disease infection and infected persons. After listing the similar genomes by scanning 100,000 or more genomes with tetra-nucleotide frequency obtained from the genome nucleotide sequence information of a target strain, a maximum likelihood tree is established by using the SNP searched by genome alignment to draw a phylogenetic tree with metadata such as sources, geographic information of strains, etc. The drawn phylogenetic tree may be applied to various fields such as tracking down the sources of strains, and the like in addition to strain identification.

In order to establish a method for constructing a gene set being suitable for the purpose, the present inventors have developed a technology to provide a method for obtaining information of reference microorganism for analyzing the genome of a sample microorganism using TNF and/or SNP.

Hereinafter, the present invention will be described in more detail.

The present invention relates to a method for obtaining information of reference microorganism for analyzing the genome of a sample microorganism, comprising selecting at least one of reference microorganisms having high correlation with the sample microorganism that is a target microorganism to be analyzed, by using whole genome nucleotide sequence and a genomic TNF of a microorganism.

In one specific embodiment, the present invention relates to a method for obtaining information of reference microorganism for analyzing the genome of a sample microorganism, comprising

providing a genomic tetra-nucleotide frequency (TNF) of a sample microorganism,

providing a genomic TNF of a reference microorganism,

selecting the reference microorganism based on correlation of genomic TNFs, by comparing genomic TNFs of the sample microorganism and the reference microorganism, and

obtaining information of the selected reference microorganism.

The sample microorganism means a microorganism to be analyzed.

In another embodiment, when the sample microorganism is a microorganism suspected of a pathogenic microorganism, it relates to a method for obtaining reference microorganism information for analyzing the genome of the sample microorganism, comprising

providing a genomic TNF of a sample microorganism,

providing a genomic TNF of a reference microorganism,

selecting the reference microorganism based on correlation of the genomic TNFs, by comparing the genomic TNFs of the sample microorganism and the reference microorganism, and

obtaining information of the selected reference microorganism.

Herein, the provision of a genomic TNF in the whole genome nucleotide sequence information of a sample microorganism is to analyze the genomic TNF in the whole genome nucleotide sequence of the sample microorganism to be analyzed.

The term “Tetra-nucleotide frequency (TNF)” means a DNA sequence fragment consisting of 4 bases (for example, AGTC, TTGG). TNFs of microbial genomes show a significant difference at the species level of microorganisms, and the TNF analysis can identify that a gene is derived from a genome of the same species or genus similar thereto with high probability. Because the TNF analysis is performed at sufficiently high processing speed, it is suitable to be used as a search engine of a large-scale genome database.

In one embodiment of the present invention, in order to obtain a genome with high correlation with a genomic TNF of a reference microorganism to be compared from database, it can be analyzed like the following method.

The method for obtaining a TNF of a microorganism may be performed by the method known in the prior art (Noble et al., 1998, Electrophoresis), and for example, the TNF of microorganism may be obtained by calculating a Z-score using the following Equation 1.

$$Z ( n 1 n 2 n 3 n 4 ) = \frac{N ( n 1 n 2 n 3 n 4 ) - E ( n 1 n 2 n 3 n 4 )}{\sqrt{var ( N ( n 1 n 2 n 3 n 4 ) )}} \qquad [ Equation 1 ]$$

In the equation, n1n2n3n4 is the combination of each nucleotide, and N(n1n2n3n4) is an actually-observed frequency of the nucleotide combination in a sample genome, and E(n1n2n3n4) is an expected frequency, and var(N(n1n2n3n4)) is variance.

The value of the expected frequency, E(n1n2n3n4) may be obtained using the following Equation 2.

$$E ( n 1 n 2 n 3 n 4 ) = \frac{N ( n 1 n 2 n 3 ) N ( n 2 n 3 n 4 )}{N ( n 2 n 3 )} \qquad [ Equation 2 ]$$

The value of the variance, var(N(n1n2n3n4)) may be obtained using the following Equation 3.

$$var ( N ( n 1 n 2 n 3 n 4 ) ) = E ( n 1 n 2 n 3 n 4 ) * \frac{[ N ( n 2 n 3 ) - N ( n 1 n 2 n 3 ) ] [ N ( n 2 n 3 ) - N ( n 2 n 3 n 4 ) ]}{{N ( n 2 n 3 )}^{2}} \qquad [ Equation 3 ]$$

The genome TNF may mean a TNF value obtained according to the method in the whole genome of a microorganism.

The reference microorganism may mean a microorganism analyzed by databased genome nucleotide sequence information, and the genome nucleotide sequence information of the reference microorganism may be stored in the database, and the TNF of the reference microorganism may be analyzed and stored according to the method for obtaining a genome TNF, but not necessarily limited thereto.

Herein, the provision of the whole genome nucleotide sequence and genome TNF of the reference microorganism may mean providing information including a TNF analyzed using the same method as the method of obtaining the whole genome nucleotide sequence information and the genome TNF of the reference microorganism.

All genomes used herein use for example, genome database developed by Chunlab Inc., and the genome database may use those stored in EzBioCloud.

Herein, the selecting of the reference microorganism based on the correlation of genome TNFs, by comparing genomic TNFs of the sample microorganism and reference microorganism may mean selecting in order of high correlation coefficient of the genomic TNF of the reference microorganism to the genomic TNF of the sample microorganism, by comparing the TNF of the sample microorganism and the TNF of the reference microorganism. The selected reference microorganism may be a microorganism or microorganism group including one or more of microorganisms.

The correlation of a genomic TNF of a reference microorganism to a genomic TNF of a sample microorganism may be obtained using Pearson correlation coefficient (PCC), but not limited thereto.

The correlation may be a Pearson correlation coefficient (PCC) value obtained using the following Equation 4.

$$r = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}} \qquad [ Equation 4 ]$$

In the Equation 4, r is a PCC value, and n is 256 in {x1, . . . , xn} and {y1, . . . , yn}, and {x1, . . . , xn} is a z-score value of a sample microorganism, and {y1, . . . , yn} is a z-score of a reference microorganism.

The PCC analyzed using a z-score value of a TNF is a value in the range of 0 to 1. As the closer the PCC value is to 1, the higher the phylogenetic similarity of the reference microorganism is to the sample microorganism.

The obtaining of information of the selected reference microorganism may provide taxonomic name information of the selected reference microorganism.

Herein, the obtaining of information of the selected reference microorganism may align the whole genome nucleotide sequence of the selected reference microorganism with the whole genome nucleotide sequence of the sample microorganism, analyze the number of SNP regions, and provide information on the phylogenetic similarity of the selected reference microorganism to the sample microorganism based on the number of SNP regions, but not limited thereto.

Herein, the term “Single nucleotide polymorphism (SNP)” refers to a genetic modification or variation showing a difference in one nucleotide (A, T, G, C) in the DNA sequence. In case of bacteria or viruses, it is used for searching the pathogenicity and antibiotic resistance in the gene unit, or it is widely used in fields such as pathological purposes or evolutionary researches through strain typing.

In the step of aligning the whole genome nucleotide sequence of the sample microorganism with the whole genome nucleotide sequence of the selected reference microorganism and analyzing the number of SNP regions, the alignment of the whole genome nucleotide sequence of the sample microorganism with the whole genome nucleotide sequence of the selected reference microorganism may use a genome alignment program such as MUMmer or Mauve, but not limited thereto.

The SNP may be obtained by calculating after performing alignment using a genome alignment tool such as MUMmer or Mauve, or may be obtained by using tools such as GATK program mapping a raw read obtained directly from NGS sequencing to the reference. However, oftentimes, the genome information disclosed in public database includes only assembled genome information without a raw read data. Therefore, it is difficult to obtain a SNP value using GATK, when the subject microorganisms to be analyzed include the genomes of all microorganisms, like the present invention.

The determination of the similarity order of genome nucleotide sequence information of the selected reference microorganism based on the number of SNP regions may determine the higher order of similarity of genome nucleotide sequence to the sample microorganism, as the number of SNP regions analyzed by the method is smaller, and may determine the lower order of similarity of genome nucleotide sequence to the sample microorganism, as the number of SNP regions is higher, as the lower order of similarity of the sample microorganism and genome nucleotide sequence.

Specifically, in the method of the present invention, the method for selecting a group of reference microorganisms is performed by calculating the tetra-nucleotide frequency (TNF) value of the sample microorganism, and comparing the genomic TNF value of the reference microorganism based on the calculated genomic TNF value of the sample microorganism. It may further comprise secondary selection based on SNP region analysis to form a sub-group of reference microorganism, by additionally performing SNP region analysis for the group of the selected reference microorganisms.

The information of a reference microorganism may include metabolic properties, biological behaviors of a microorganism, and the like. In addition, the selection of a reference microorganism includes selection based on a genome TNF and/or selection based on the result of SNP region analysis. Preferably, the primary selection using the genome TNF criteria may be performed and then the secondary selection based on the result of SNP region analysis may be performed. Accordingly, the selected reference microorganism may be selected by performing the selection by TNF value, the selection by SNP region analysis, or both of the selection by TNF value and the selection by SNP region analysis. A device or system for obtaining information of reference microorganism for analyzing a sample microorganism according to the present invention may comprise a criteria providing system which provides the TNF criteria and/or the result of SNP region analysis; a scoring system or sorting system which gives a score or rank to the reference microorganism according to the selection; and a grouping or sub-grouping system which forms a group or sub-group of the reference microorganisms according to the score and/or rank. The device or system for obtaining information of reference microorganism for analyzing a sample microorganism according to the present invention may further comprise a system of analyzing or producing information of a sample microorganism according to the information of the reference microorganism group or sub-group.

Herein, the method of selecting in order of high correlation with the genomic TNF value of the sample microorganism and determining the order of similarity of genome nucleotide sequence based on the number of SNP regions, has significantly high speed and significantly high accuracy at the genus level, compared to the similar genome selecting analysis of aligning using 16S rRNA and BLAST.

Because the method for obtaining information of a reference microorganism for analyzing the genome of a sample microorganism using TNF-SNP in the present invention adopts the nucleotide level (both TNF and SNP) as a molecular level, it is not affected by the presence or the absence of a specific gene(s) and can analyze with being independent of a specific gene. The method of present invention has a difference in the method for obtaining information of a reference microorganism for analyzing the genome of a sample microorganism using 16S rRNA or a housekeeping gene can analyze only when a specific target gene is acquired.

The step of providing information of the selected reference microorganism may mean providing information of the selected reference microorganism after selecting the reference microorganism in order of high correlation with the genome TNF value of a sample microorganism by the method, and the information of a reference microorganism may include metadata, but not limited thereto. The information of reference microorganism may include metabolic properties, biological behaviors of a microorganism, and the like. In addition, the selection of a reference microorganism includes the selection by the genome TNF and/or the selection by the result of SNP region analysis. Preferably, the primary selection using the genome TNF criteria may be performed and then the secondary selection based on the result of SNP region analysis may be performed. Thus, the reference microorganism may be selected by performing the selection by TNF value, the section by result of SNP region analysis, or both the selection by TNF value and the section by result of SNP region analysis.

The metadata means data describing data, and the metadata used herein refers to additional information obtained by performing the TNF and SNP for the genome. The additional information herein may include at least one selected from the group consisting of pathogenicity, environmental information on the source, year of discovery, country of discovery and related literature information (for example, information on the published academic journal) of a microorganism. The researchers can construct a genomic set required for themselves using metadata freely, and for example, can obtain information required for a follow-up survey using metadata.

By using the information of a reference microorganism for analyzing the genome of a sample microorganism, a comparative genome set consisting of the sample microorganism and reference microorganism may be produced. In one embodiment of the present invention, a group (or combination) of the selected reference microorganisms may be obtained using a genomic TNF and a sub-group (or sub-combination) of the selected reference microorganisms may be obtained using genomic TNF and SNP.

FIG. 1 is a schematic drawing of establishment of the comparative genome set by comparing a TNF of a sample genome with TNF database in EzBioCloud to select 250 strains having similar genomes, sorting 250 strains in order of high similarity based on the SNP by extracting an SNP in the genomes, and then forming a comparative genomic set by adding metadata of EzBioCloud to 250 sorted genomes according to an example of the present invention, in case of directly selecting the genomes of strains from the list for performing the comparative genome analysis.

A core genome, a pan-genome or a genomic island may be analyzed by using the comparative genome set containing the sample microorganism and reference microorganism obtained in the present invention. The term, “core genome” refers to a gene which is common in a given set of genomes.

The term, “pan-genome” refers to the sum of genes possessed by a given set of genomes.

The term, “genomic island” refers to a combination of genes which move at a molecular (DNA) level as a direct proof of horizontal gene transfer in a microbial genome.

Herein, the information of reference microorganisms including the whole genome nucleotide sequence and genomic TNF may be stored in a computer recording medium, and a cloud sever of the computer recording medium may be EzBioCloud (ezbiocloud.net) or BIOiPLUG (bioiplug.com), but not limited thereto.

An embodiment of the present invention provides a computer reading method for obtaining information of a reference microorganism for genome analysis of a sample microorganism for executing steps of the method.

The computer reading method may mean analyzing data on a computer using a computer program stored in a computer readable storage medium to execute each step described above for performing the method described in the present invention, but not limited thereto.

The method and information described herein provides a computer program stored in a computer readable storage medium for executing the steps of the method capable of executing the steps described above. The computer program stored in a computer readable storage medium may be combined with hardware. The computer program stored in a computer readable storage medium is a program for executing the steps on a computer, and then, all the aforementioned steps may be executed by one program, or may be executed by two or more of programs. The computer readable storage program or software may be transferred to a computer device by all the known transfer method including for example, on a communication channel such as a telephone line, Internet, wireless connection or the like, or through a transportable medium such as a computer readable disk, flash drive or the like.

In addition, one embodiment of the present invention relates to a storage medium recording a computer readable program for executing the method for obtaining information of a reference microorganism for genome analysis of a sample microorganism using the method.

The storage medium recording a computer readable program may include both computer storage medium and communication medium.

The computer storage medium includes all of volatile and non-volatile, and removable and non-removable media materialized by any method or technique for storage of information such as computer readable commands, data structures, program modules or other data. For example, the computer storage medium may be one or more selected from RAM, ROM, EEPROM, flash memory (e.g. USB memory, SD memory, SSD, CF memory, xD memory, etc.), magnetic disk, laser disk or other memory, CD-ROM, DVD (Digital versatile disk) or other optical disk, magnetic cassette, magnetic tape, magnetic disk storage device or other magnetic storage device, or all media which is capable of being used to store desired information and is accessible by a computer, but not limited thereto.

The communication medium typically comprises computer readable commands, data structures, program modules or other data of altered data signals such as a carrier wave, or other transmission mechanism, and comprises any information transmission medium. For example, the communication medium may be one or more selected from wired media such as wired network or direct-wired connection, and wireless media such as acoustic media, RF, infrared and other wireless media.

One or more combinations of the aforementioned media may be included in the range of the computer readable medium. The example of the computer readable medium according to one embodiment of the present invention is illustrated in FIG. 5, and for example, as one component of a computer system (500), the computer system may comprise one or more processors (510), one or more of computer readable storage media (530) and a memory (520).

The cloud computing means an Internet-based (cloud) computing technique. It is a web-based software service that places programs on a utility data server on the Internet and then loads them into a computer or mobile phone from time to time and uses them. The cloud computing may be a type of computing system that mainly performs only input/output operations through personal terminals, and performs operations such as information analysis and processing, storage, management and distribution and the like in a third space called cloud.

### Advantageous Effects

The present invention relates to a method for prepare a comparative genomics set, by analyzing a TNF of a sample genome and aligning the selected genome in order of similarity, and specifically, relates to a method for obtaining information of reference microorganism for analyzing genome of a sample microorganism, including analyzing a genomic tetra-nucleotide frequency (TNF) in the whole genome nucleotide sequence information of a sample microorganism, providing information of reference microorganisms including the whole genome nucleotide sequence information and their TNFs, selecting the reference microorganism based on correlation of the genomic TNFs by comparing the genomic TNFs of the sample microorganism and providing information of the selected reference microorganism.

The present invention relates to a method for tracing a pathogenic microorganism using SNP and metadata of microorganism, including analyzing TNF of a sample genome and sorting the selected genome in order of similarity, and specifically, relates to a method for obtaining information of a reference microorganism for follow-up study on a genome of a sample pathogen, including analyzing a genomic TNF of the whole genome nucleotide sequence information of the sample pathogen, providing information of reference microorganisms including the whole genome nucleotide sequence information and their TNFs, selecting the reference microorganisms based on the correlation with the genomic TNF of the sample microorganism by comparing the genomic TNFs of the sample pathogen and reference microorganism, finding the SNP of the selected reference microorganism, and providing information of the selected reference microorganisms.

### BRIEF DESCRIPTION OF THE DRAWINGS

FIG. 1 is a schematic drawing of selecting 250 strains having the similar genomes by comparing a TNF of a sample genome with EzBioCloud TNF database and extracting an SNP in the genomes, sorting 250 strains in order of high similarity based on the SNP, and then forming a comparative genomic set by adding the metadata in EzBioCloud to the genome of sorted 250 strains.

FIG. 2 is a drawing showing a drastic increase of the number of sequence genomes over time.

FIG. 3 is a drawing showing that a strain having a similar genome according to the method in the example of the present invention can select at a significantly rapid rate, compared to the comparative example in which a similar strain is searched using 16S rRNA of the strain genome with BLAST.

FIG. 4 is a drawing showing that the accuracy is significantly higher in case of selecting and aligning a similar strain by the method in the example of the present invention, compared to the comparative example in which a similar strain is searched using 16S rRNA of the strain genome with BLAST.

FIG. 5 illustrates an example of the computer readable medium according to one embodiment of the present invention.

FIG. 6 is a result of follow-up study for a pathogen, Klebsiella pneumoniae causing an outbreak according to one embodiment of the present invention.

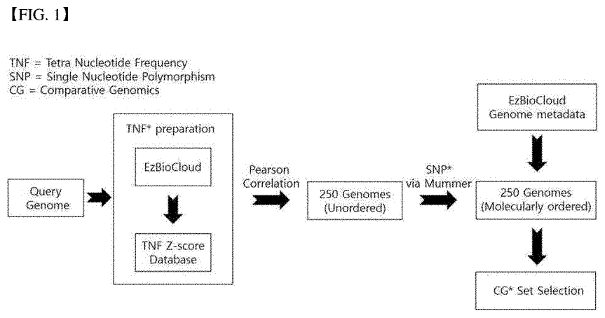

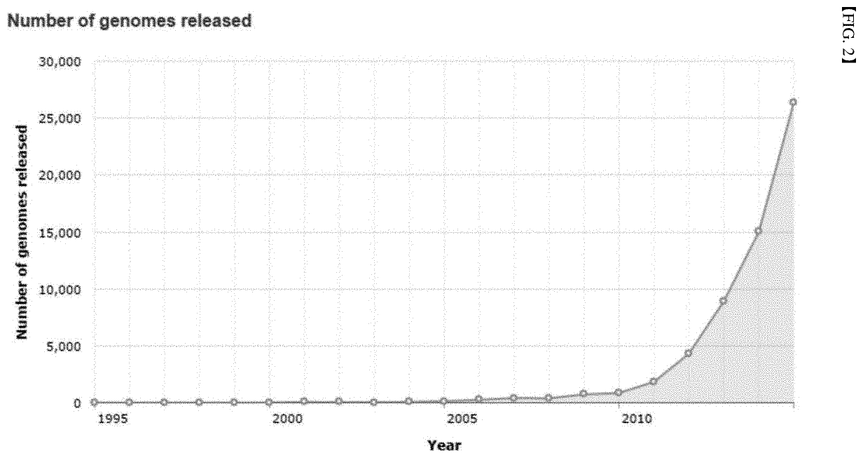

### DETAILED DESCRIPTION OF THE EMBODIMENTS

Herein, the present invention will be described in more detail by the following examples. However, these examples are intended to illustrate the present invention only, but the scope of the present invention is not limited by these examples.

All genomes used in the present examples use genome database developed by Chunlab, Inc. All genomes of EzBioCloud may be microbial genome database in which gene analysis is performed by the proven method developed by Chunlab, Inc., and the cloud server may be EzBioCloud (ezbiocloud.net) or BIOiPLUG (bioiplug.com).

### <Example 1> Selection of Similar Genomes Using TNF

1-1. Analysis of TNF for the Genome

The sample genomes were collected from sequences that were directly sequenced from NGS using samples obtained from patient or environments and random genomes from the EzBioCloud database.

The list of genomes for further comparative genomic analysis was obtained from the database that has a high correlation with the sample genome using the following methods.

As proposed by Noble et. al. in 1998, the calculation method for TNF has already been known. (Noble, P. A., Citek, R. W., & Ogunseitan, O. A. (1998). Tetranucleotide frequencies in microbial genomes. Electrophoresis, 19(4), 528-535.). Specifically, the TNF of a microorganism can be obtained by following formula which calculates a Z-score.

$$Z ( n 1 n 2 n 3 n 4 ) = \frac{N ( n 1 n 2 n 3 n 4 ) - E ( n 1 n 2 n 3 n 4 )}{\sqrt{var ( N ( n 1 n 2 n 3 n 4 ) )}} \qquad [ Equation 1 ]$$

In equation 1, n1n2n3n4 is the combination of each nucleotide, and N(n1n2n3n4) is a frequency that was observed in a sample genome, and E(n1n2n3n4) is an expected frequency, and var(N(n1n2n3n4)) is variance.

The value of the expected frequency, E(n1n2n3n4) may be obtained using Equation 2 below. The value of the variance, var(N(n1n2n3n4)) may be obtained using Equation 3 below.

$$\begin{matrix}E ( n 1 n 2 n 3 n 4 ) = \frac{N ( n 1 n 2 n 3 ) N ( n 2 n 3 n 4 )}{N ( n 2 n 3 )} & [ Equation 2 ] \\ var ( N ( n 1 n 2 n 3 n 4 ) ) = E ( n 1 n 2 n 3 n 4 ) * \frac{[ N ( n 2 n 3 ) - N ( n 1 n 2 n 3 ) ] [ N ( n 2 n 3 ) - N ( n 2 n 3 n 4 ) ]}{{N ( n 2 n 3 )}^{2}} & [ Equation 3 ]\end{matrix}$$

1-2. Construction of TNF Database

All genomes were targeted for the calculation of TNF using the method described in Example 1-1. The database was generated to store the calculated TNF, and it was provided in the EzbioCloud database by ChunLab Currently, the EzBioCloud database contains TNF data of 92,802 genomes.

1-3. Selection of Similar Genomes

The strains having similar genomes can be selected by calculating a TNF value of a sample microorganism by using the method described in Example 1-1, comparing TNF value of a sample microorganism with the TNF of genomes stored in the EzBioCloud database by ChunLab in Example 1-2, and arranging them from high TNF similarity species to low TNF similarity species. Specifically, 250 microbial strains having high genome similarity from EzBioCloud database were ordered based on the Pearson correlation coefficient (PCC), where the genomes with high PCC were listed on top. The Pearson correlation coefficient (PCC) value can be calculated by Equation 4 below.

$$r = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}} \qquad [ Equation 4 ]$$

In the Equation 4, r is a PCC value, and n is 256 in {x1, . . . , xn} and {y1, . . . , yn}, and {x1, . . . , xn} is a z-score value of a sample microorganism, and {y1, . . . , yn} is a z-score of a reference microorganism.

### <Example 2> Genome Alignment in Order of Similarity Using SNP Markers

2-1. Extraction of SNP Markers in Selected Genomes

In order to arrange 250 genomes selected using the TNF by the method of Example 1 in order of high similarity, SNP regions were extracted by the following method.

Specifically, in order to extract SNP regions of the selected 250 genomes, the number of SNP regions in the selected 250 genomes was confirmed by applying a genome of an unknown microorganism and 250 genomes selected by the method of Example 1, respectively.

2-2. Alignment of Genome Using SNP Regions

The similarity of genomes is high as the number of SNP regions is small between general genomes to be compared. Thus, the similarity between genomes can be identified by determining the number of SNP regions between genomes. By arranging the genomes sequentially from small number of SNP regions in Example 2-1 to high number, the genomes selected in Example 1 were accurately arranged in order of genome similarity from the genomes with high similarity to genomes with low similarity.

### <Example 3> Addition of Metadata to Selected and Arranged Genomes

Researchers conducting comparative genomics may have different genomes to be selected and required additional data depending on their direction and purpose of the research. For example, when they want to research on the trends of evolutionary changes by certain environmental factors, apart from the information of genomes with high similarity to a target genome, additional data such as places or locations where the genome is discovered and the like may be required. According to the above need, a comparative genomics data set may be prepared by adding additional data to the genome information similar to a sample microorganism.

In order to prepare the comparative genomics set, additional data of the corresponding microorganism can be identified in EzBioCloud of Chunlab, to 250 genome information in order of genome similarity to the target microorganism prepared by the method of Examples 1 to 2. The additional data of the corresponding microorganism stored in EzBioCloud (Chunlab) include pathogenicity, environmental information about source (for example, soil, food, host, etc.), year of discovery, country of discovery or information about the published academic journal (related articles) and the like.

Since researchers can obtain additional data required for their research in addition to genome information selected by the high genome similarity and arranged in order of similarity, through the technique for preparing a comparative genomics set by adding the additional data, there is an advantage of obtaining good quality data without additional information search.

### <Example 4> Selection of Similar Genomes Using TNF

By using the substantially same method as the Examples 1-1, 1-2 and 1-3, the TNF of genomes was analyzed, and the TNF database of EzBioCloud was constructed and similar genomes were selected. However, in the selecting similar genomes, 30 microbial strains with the most similar genomes were selected in EzBioCloud database.

As an unknown sample microorganism used for drawing the experimental result of FIG. 6, the genome of the strain derived from the first patient in the article epidemiologically examining an outbreak occurred in NIH hospital in 2011 was received from EzBioCloud to analyze.

### <Example 5> Alignment of Genome in Order of Similarity Using SNP Regions

5-1. Extraction of SNP Markers in Selected Genomes

In order to arrange 30 genomes selected using a TNF by the method of Example 4 in order of high similarity, SNP regions were extracted by the following method.

Specifically, in order to extract SNP regions in selected 30 genomes, SNPs present in the selected 30 genomes were found by applying a genome of an unknown sample microorganism and 30 genomes selected by the method of Example 4, respectively, to MuMmer genome alignment program.

### 5-2. Drawing of a Phylogenetic Tree Using SNP Regions

After obtaining any sequence by extracting the SNP confirmed in Example 5-1 from each genome and arranging them by location, a phylogenetic tree using Maximum likelihood was inferred using RAxML program. The phylogenetic tree obtained by the result was shown in FIG. 6.

FIG. 6 shows the strain isolated from the firstly infected patient causing an outbreak of Klebsiella pneumoniae in U.S. NIH hospital in 2011, where the stain is indicated by a red arrow in FIG. 6. As a result of applying the method of the present invention, the strain in FIG. 6 could be found. The present method can inferred that the sample strain is Klebsiella pneumoniae with only a genome nucleotide sequence without additional information, and 14 strains among 29 strains not including the sample microorganism were actually collected in the same hospital. Thus, it can be utilized for identify the infection route, when it is utilized together with information such as patient moving route and time required for the definite diagnosis of infection and the like. In case that the genome nucleotide sequences were analyzed completely, the analysis time was 6 minutes and 40 seconds.

### <Example 6> Addition of Metadata to Selected and Arranged Genomes

To tracking a pathogen effectively, in addition to the analysis of the genome similarity used in Examples 4 and 5, metadata such as sampling places and time of the pathogen used for the analysis and the like are certainly required.

In order to prepare the comparative genomics set, in addition to information of 30 genomes in order of genome similarity to the test microorganism prepared by the method of Examples 4 to 5, the metadata of the corresponding microorganism stored in EzBioCloud (Chunlab) were disclosed in Table 1. The metadata of the corresponding microorganism stored in EzBioCloud (Chunlab) include pathogenicity, environmental information on source (for example, soil, food, host, etc.), year of discovery, country of discovery or information about the published journal (related articles) of the corresponding microorganism and the like. The metadata of strains used for analysis in FIG. 6 were shown in the following Table 1.

TABLE 1

| Strain | | Biosample | | | | |
|---|---|---|---|---|---|---|
| name | Publication | Accession No | Year | Source | Geo origin | Country |
| KPNIH2 | 22914622 | SAMN01057609 | 2011 | Host: Homo sapiens | Bethesda, MD | United States |
| KPNIH5 | 22914622 | SAMN01057607 | 2011 | Host: Homo sapiens | Bethesda, MD | United States |
| KPNIH6 | 22914622 | SAMN01057608 | 2011 | Host: Homo sapiens | Bethesda, MD | United States |
| KPNIH9 | 22914622 | SAMN01057613 | 2011 | Host: Homo sapiens | Bethesda, MD | United States |
| KPNIH12 | 22914622 | SAMN01057621 | 2011 | Host: Homo sapiens | Bethesda, MD | United States |
| KPNIH17 | 22914622 | SAMN01057646 | 2011 | Host: Homo sapiens | Bethesda, MD | United States |
| KPNIH20 | 22914622 | SAMN01057650 | 2011 | Host: Homo sapiens | Bethesda, MD | United States |
| KPNIH4 | 22914622 | SAMN01057606 | 2011 | Host: Homo sapiens | Bethesda, MD | United States |
| KPNIH7 | 22914622 | SAMN01057711 | 2011 | Host: Homo sapiens | Bethesda, MD | United States |
| KPNIH10 | 22914622 | SAMN01057614 | 2011 | Host: Homo sapiens | Bethesda, MD | United States |
| KPNIH14 | 22914622 | SAMN01057640 | 2011 | Host: Homo sapiens | Bethesda, MD | United States |
| KPNIH1 | 22914622 | SAMN01057611 | 2011 | groin(Host: Homo sapiens) | Bethesda, MD | United States |
| KPNIH8 | 22914622 | SAMN01057612 | ** | Host: Homo sapiens | Bethesda, MD | United States |
| KPNIH11 | 22914622 | SAMN01057620 | ** | Host: Homo sapiens | Bethesda, MD | United States |
| KPNIH16 | 22914622 | SAMN01057641 | ** | Host: Homo sapiens | Bethesda, MD | United States |
| BIDMC60 | ** | SAMN02581281 | 2013 | Clinical(peritoneal fluid) | Boston, MA | United States |
| CHS 56 | ** | SAMN02581358 | 2013 | Clinical(urine) | North Carolina | United States |
| MGH 51 | ** | SAMN02581383 | 2013 | Clinical(urine) | Boston | United States |
| MGH 79 | ** | SAMN02581252 | 2013 | Clinical(urine) | Boston | United States |
| CHS114 | ** | SAMN03280257 | 2014 | Host: Homo sapiens | ** | United States |
| CHS207 | ** | SAMN03280348 | 2014 | Host: Homo sapiens | ** | United States |
| KpVA-5 | ** | SAMEA3108421 | ** | ** | ** | ** |
| KpVA-7 | ** | SAMEA3108423 | 2011 | ** | ** | ** |
| KpVA-8 | ** | SAMEA3108424 | 2013 | ** | ** | ** |
| KpVA-10 | ** | SAMEA3108426 | ** | ** | ** | ** |
| KpVA-11 | ** | SAMEA3108427 | 2013 | ** | ** | ** |
| KpVA-13 | ** | SAMEA3108429 | | ** | ** | ** |
| KpVA-14 | ** | SAMEA3108430 | 2013 | ** | ** | ** |
| KPNIH36 | ** | SAMN03701676 | 2013 | ** | ** | United States |
| KPNIH34 | 25232178 | SAMN05346258 | 2012 | perirectal(Host: Homo sapiens) | ** | ** |
| blood sample 2 | ** | SAMN05149976 | 2010 | blood(Host: Homo sapiens) | Basel | Switzerland |

### <Comparative Example 1> Selection of Similar Genomes Using 16S rRNA

10 genomes were randomly selected from EzBioCloud genome database, and the 16S rRNA sequence information of genomes of the corresponding 10 microorganisms was obtained from EzBioCloud genome database. The information of 10 microorganisms was disclosed in the following Table 2. The similarity of the 16S rRNA sequence information of the genomes of 10 microorganisms and the 16S rRNA sequence information of other genomes present in EzBioCloud genome database was confirmed using BLAST, and 250 genomes with high similarity shown in the result were selected.

### <Example 7> Analysis of Selection Rates of Similar Genomes by TNF Analysis

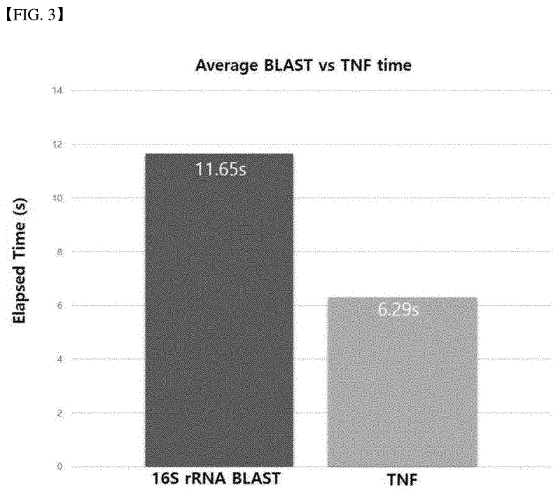

The following Table 2 represents the required time measured during the process of selecting 250 genomes with high similarity according to the method of Example 1 and Comparative Example 1 for 20 genomes randomly selected in EzBioCloud. TNFs were calculated in the sample genome according to Example 1, and the time of selecting 250 genomes was calculated by comparison with TNF database of EzBioCloud. The database was produced by extracting 16S rRNA from the genomes in EzBioCloud according to Comparative Example 1, and was aligned with BLAST for 16S rRNA of the sample microorganism to select 250 16S rRNA. The time needed for the analysis was measured except for the extraction of 16S rRNA from genomes. As a result of comparing and analyzing the time required to select similar genomes, it could be confirmed that the average time needed for performing the method of Example 1 selecting the similar genomes using TNF was 1.9-fold faster that of the method of selecting the similar genomes using 16S rRNA in Comparative example 1. The comparison of the average time in Table 2 was represented in FIG. 3.

TABLE 2

| | Time | Time | |
|---|---|---|---|
| | required | required | |
| | for BLAST | for TNF | Fold of |
| | analysis | analysis | required |
| Strain | (sec) | (sec) | time |
| Escherichia coli | 8.376 | 5.609 | 1.493314 |
| Burkholderiapseudomallei | 11.369 | 6.24 | 1.821955 |
| Vibrio parahaemolyticus | 10.675 | 5.546 | 1.924811 |
| Galbibacter marinus | 14.493 | 5.488 | 2.640853 |
| Mycobacterium tuberculosis | 7.519 | 6.467 | 1.162672 |
| Enterobacter xiangfangensis | 9.078 | 7.501 | 1.210239 |
| Frankia casuarinae | 11.591 | 5.546 | 2.089975 |
| Pseudomonas aeruginosa | 10.088 | 5.474 | 1.842894 |
| Brucella canis | 11.982 | 7.539 | 1.589335 |
| Methanosarcina mazei | 11.696 | 8.261 | 1.415809 |
| Tsukamurella tyrosinosolvens | 11.701 | 5.436 | 2.152502 |
| Staphylococcus epidermidis | 7.025 | 5.283 | 1.329737 |
| Streptococcus pneumoniae | 8.398 | 6.933 | 1.211308 |
| Pseudomonas avellanae | 12.679 | 5.585 | 2.270188 |
| Leptospira interrogans | 18.292 | 5.781 | 3.164158 |
| Paracoccus yeei | 17.098 | 5.452 | 3.136097 |
| Acinetobacter baumannii | 11.658 | 5.363 | 2.173783 |
| Haemophilus influenzae | 11.974 | 5.067 | 2.363134 |
| Leuconostoc fallax | 16.747 | 9.694 | 1.727563 |
| Melissococcus plutonius | 10.682 | 7.629 | 1.400184 |

### <Example 8> Analysis of Accuracy of Genome Selection Using a TNF and an SNP

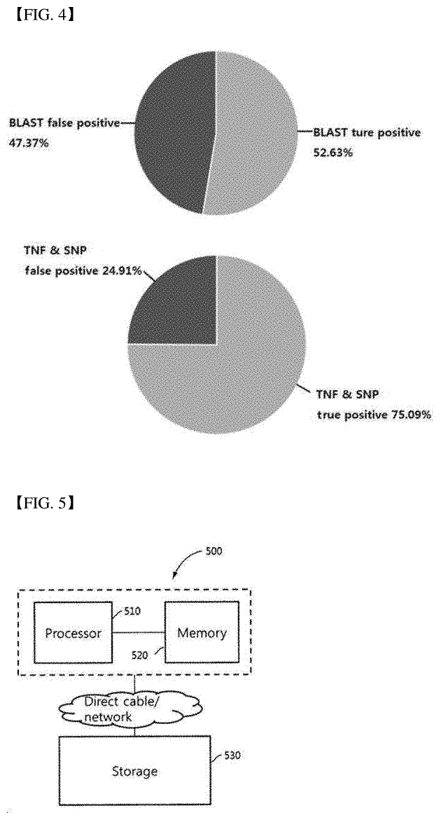

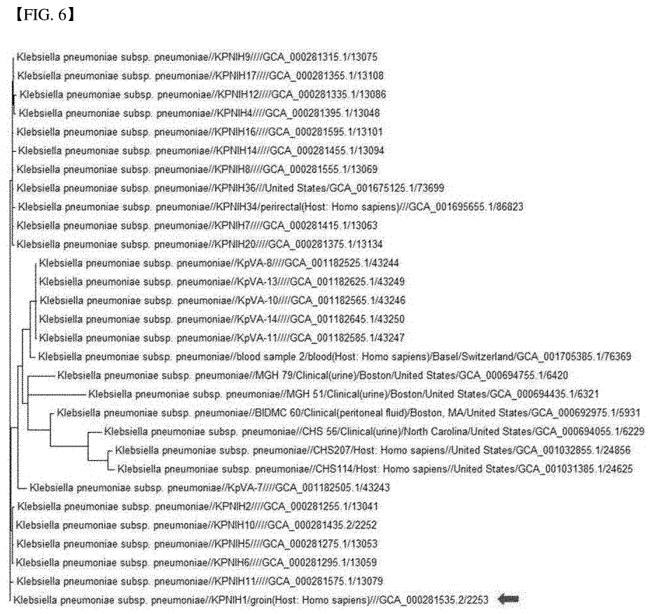

For 10 genomes randomly selected from EzBioCloud genome database, 250 similar genomes were selected, and were arranged in order of similarity by the method of Examples 1 to 2 and Comparative example 1. The accuracy of selecting the genomes was compared, and the result of comparative accuracy analysis for the selection was shown in FIG. 4.

Specifically, in order to test the accuracy of selecting 250 similar genomes and arranging them in order of genome similarity according to the method of Examples 1 to 2 and Comparative example 1 at a strain level, the result of analyzing the similar genomes using the whole genome according to OrthoANI method was set as the reference value.

As the OrthoANI method had a characteristic of analyzing the whole genomes by fragmenting them (1024 base pair) and analyzing with the parts that two genomes shared, the result of analyzing similar genomes using the OrthoANI method was set as the reference value for analysis at a strain level.

The accuracy of the strains selected and arranged in order of similarity using TNF and SNP analysis of Examples 1 to 2 and the accuracy of 16s rRNA BLAST method of Comparative Example 1 were analyzed and shown in FIG. 4 together with the reference value.

As a result, it could be confirmed that the method of performing BLAST analysis using 16s rRNA extracted from the whole genomes according to Comparative Example 1 had a lower accuracy at a strain level by about 22.5%, compared to the method using TNF-SNP of Example 2 of the present invention. From the analysis result, it could be seen that the method for selecting and arranging similar strains using TNF-SNP of the present invention had a significantly higher accuracy at a strain level, compared to the method for performing BLAST analysis using 16S rRNA extracted from the whole genomes according to Comparative example 1.

1. A method for obtaining information on a reference microorganism for analyzing genome of the sample microorganism, wherein the method comprises:
providing a tetra-nucleotide frequency (TNF) of the sample microorganism’ genome,
providing a TNF of a reference microorganism's genome;
comparing the TNFs of the sample microorganism and the reference microorganism and selecting reference microorganism based on their correlation value of TNF; and
obtaining information of the selected reference microorganism.

2. The method of claim 1, wherein the step of selecting the reference microorganism is made based on the order of high correlation coefficient of the TNF of the reference microorganism’ genome to the genomic TNF of the sample microorganisms.

3. The method of claim 2, wherein the correlation coefficient of the TNF of the reference microorganism’ genome to the TNF of the sample microorganism's genome is obtained using Pearson correlation coefficient (PCC).

4. The method of claim 1, wherein the method further comprises a step of conducting secondary selection from reference microorganisms based on the number of single nucleotide polymorphism (SNP) by aligning a genome nucleotide sequence of the selected reference microorganism with a genome nucleotide sequence of the sample microorganism to analyze the number of SNPs.

5. The method of claim 1, wherein the step of obtaining information of the selected reference microorganisms provides at least one information selected from the group consisting of taxonomically valid name, phylogenetic similarity, pathogenicity, environmental information on sampling site, year of discovery, country of discovery, and related literature information, for the selected reference microorganisms.

6. The method of claim 1, wherein a core gene, a genomic island or a pan-genome of the sample microorganism is analyzed using the information of the reference microorganisms obtained for analyzing the genome of sample microorganism.

7. The method of claim 5, wherein the method further comprise tracking down the pathogenicity of the sample microorganism by using the pathogenic information of the selected reference microorganism.

8. The method of claim 1, wherein the TNF of genome is calculated using a Z-score.

9. The method of claim 1, wherein the information of the reference microorganisms including genome nucleotide sequence or TNF is recorded in the computer recording media.

10. The method of claim 9, wherein the computer recording media is located in a cloud server.

11. A computer reading method for obtaining information on reference microorganisms for analyzing genome of the sample microorganism using the method of claim 1.

12. A computer storage media, which records a computer-readable program for executing a method for obtaining information on reference microorganisms for analyzing genome of the sample microorganism using the method of claim 1.
